(19) United States
(12) Patent Application Publication
(10) Pub. No.: US 2011/0123622 A1
(43) Pub. Date: May 26, 2011

(54) OPHTHALMIC FORMULATION AND METHOD OF MANUFACTURE THEREOF

(75) Inventors: Kenneth L. Avery, Merrimack, NH (US); Harun Takruri, Newport Beach, CA (US)

(73) Assignee: Inotek Pharmaceuticals Corporation, Lexington, MA (US)

(21) Appl. No.: 12/912,054

(22) Filed: Oct. 26, 2010

Related U.S. Application Data
(60) Provisional application No. 61/254,923, filed on Oct. 26, 2009.

(51) Int. Cl. A61K 9/14; A61P 27/02; A61K 31/7076

(52) U.S. Cl. 424/489; 514/46

(57) ABSTRACT

Provided herein is an ophthalmic formulation that comprises a fine particle of an A₁ agonist in an aqueous suspension and a manufacturing process thereof. More specifically, provided herein is a topically applied ophthalmic aqueous suspension which is obtainable by suspending a fine particle of an A₁ agonist in a surfactant and preservative; a method of reduction of intraocular pressure using the formulation and a manufacturing process of the aqueous suspension thereof.

### RELATED APPLICATION

This application claims priority to U.S. Provisional Application No. 61/254,923, Attorney Docket No. ITJ-045-1, filed Oct. 26, 2009, titled “OPHTHALMIC FORMULATION AND METHOD OF MANUFACTURE THEREOF.” The contents of any patents, patent applications, and references cited throughout this specification are hereby incorporated by reference in their entireties.

### TECHNICAL FIELD

Provided herein is an ophthalmic formulation that comprises a fine particle of an A₁ agonist in an aqueous suspension and a manufacturing process thereof. More specifically, provided herein is a topically applied ophthalmic aqueous suspension that is obtainable by suspending a fine particle of an A₁ agonist in a surfactant and preservative; a method of reduction of intraocular pressure using the formulation and a manufacturing process of the aqueous suspension thereof.

### BACKGROUND

In copending patent application U.S. Ser. No. 12/771,289, the Applicant has shown clinically significant reduction of intraocular pressure using an A₁ agonist in human subjects having glaucoma. The specification of U.S. Ser. No. 12/771,289 is herein incorporated in its entirety as if individually set forth.

The specification of U.S. Ser. No. 12/771,289 describes a formulation comprising 1 part of an A₁ agonist to 20 parts Hydroxypropyl β-Cyclodextrin (HPβCD) (i.e. 1:20 wt/wt) reconstituted with 0.9% Saline for Injection, USP, at concentrations indicated below.

| Clinical Dose | Compound A | Ocular Dose |
|---|---|---|
| (mcg/eye) | (mg/mL) | Volume (μL) |
| 2.5 | 0.05 | 50 |
| 7.5 | 0.15 | 50 |
| 20 | 0.40 | 50 |
| 60 | 1.2 | 50 |
| 180 | 3.6 | 50 |
| 350 | 7.0 | 50 |
| 700 | 7.0 | 2 × 50 |

A difficulty with the HPβCD formulation was that the formulation was prepared as a lyophile that needed to be reconstituted with saline prior to use. This was because the stability of the A₁ agonist was limited, and after time a by-product of Compound A would begin to be formed. While the HPβCD formulation could be used in the first clinical trials and was shown to be effective for treatment, the Applicant has sought to develop a new formulation with enhanced stability and without the need for preparation of a lyophile and subsequent reconstitution. Therefore, there has been a need to develop a stable ophthalmic formulation for delivering an A₁ agonist and a process for manufacturing the ophthalmic formulation.

### SUMMARY OF INVENTION

In a first aspect of the invention there is provided an ophthalmic formulation comprising:

(a) an aqueous suspension of fine particles of an A₁ agonist,

(b) a surfactant, and

(c) a preservative.

In one embodiment the suspension of an A₁ agonist comprises fine particles of less than 50 microns. In another embodiment the fine particles are less than 10 microns. In a further embodiment the fine particles are between 3-7 microns.

In one embodiment the A₁ agonist is selected from:

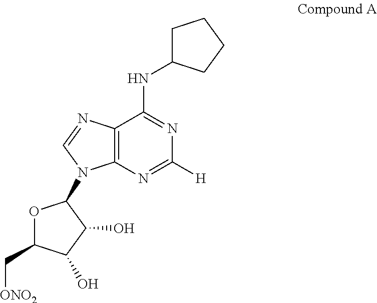

((2R,3S,4R,5R)-5-(6-(cyclopentylamino)-9H-purin-9-yl)-3,4-dihydroxytetrahydrofuran-2-yl)methyl nitrate,

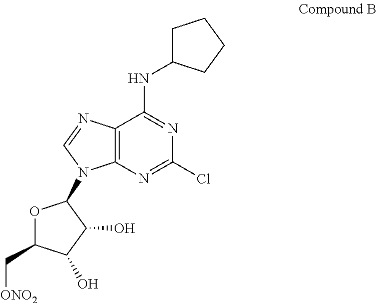

((2R,3S,4R,5R)-5-(2-chloro-6-(cyclopentylamino)-9H-purin-9-yl)-3,4-dihydroxytetrahydrofuran-2-yl)methyl nitrate,

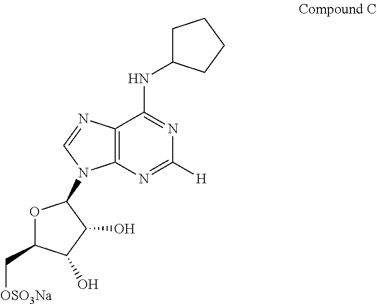

sodium ((2R,3S,4R,5R)-5-(6-(cyclopentylamino)-9H-purin-9-yl)-3,4-dihydroxytetrahydrofuran-2-yl)methyl sulfate,

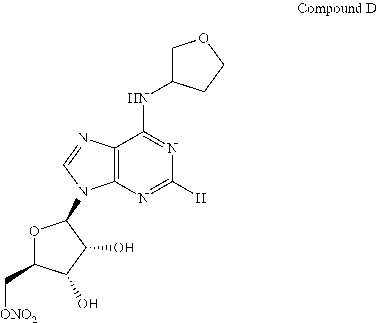

((2R,3S,4R,5R)-3,4-dihydroxy-5-(6-(tetrahydrofuran-3-ylamino)-9H-purin-9-yl)tetrahydrofuran-2-yl)methyl nitrate,

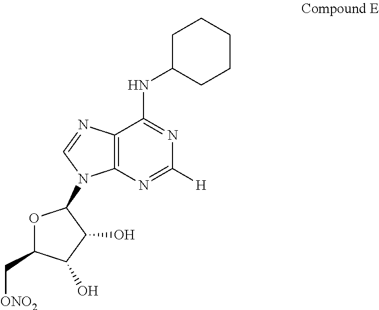

((2R,3S,4R,5R)-5-(6-(cyclohexylamino)-9H-purin-9-yl)-3,4-dihydroxytetrahydrofuran-2-yl)methyl nitrate,

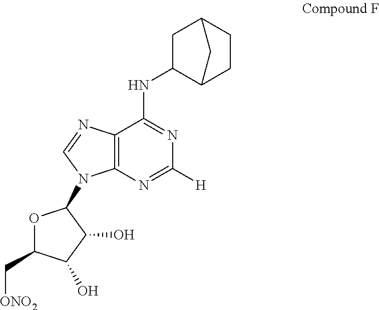

((2R,3S,4R,5R)-5-(6-(bicycle-[2.2.1]-heptan-2-ylamino)-9H-purin-9-yl)-3,4-dihydroxytetrahydrofuran-2-yl)methyl nitrate,

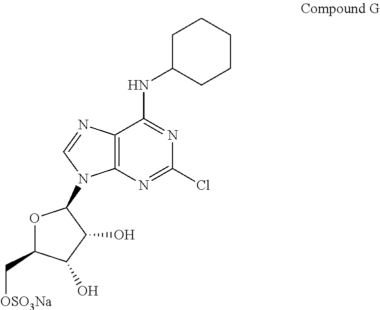

sodium ((2R,3S,4R,5R)-5-(2-chloro-6-(cyclohexylamino)-9H-purin-9-yl)-3,4-dihydroxytetrahydrofuran-2-yl)methyl sulfate, and

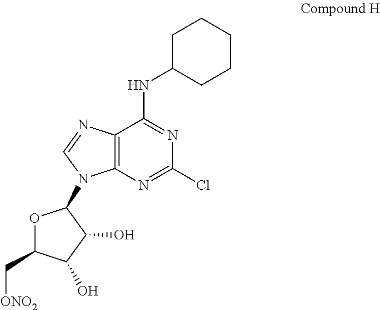

((2R,3S,4R,5R)-5-(2-chloro-6-(cyclohexylamino)-9H-purin-9-yl)-3,4-dihydroxytetrahydrofuran-2-yl)methyl nitrate.

In one embodiment, the A1 agonist is compound A

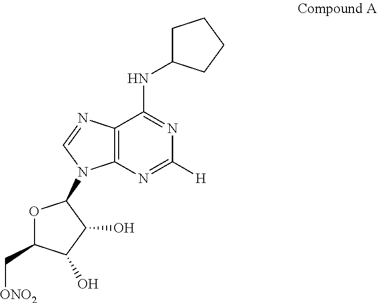

((2R,3S,4R,5R)-5-(6-(cyclopentylamino)-9H-purin-9-yl)-3,4-dihydroxytetrahydrofuran-2-yl)methyl nitrate.

In one embodiment the surfactant is selected from polysorbate 80, polysorbate 60, polysorbate 40, polysorbate 20, polyoxyl 40 stearate, poloxamers, tyloxapol, POE 35 and castor oil.

In one embodiment the preservative is selected from a quaternary ammonium salts including benzalkonium chloride, cetrimide, chlorobutanol, sorbic acid, boric acid, and any other preservatives known to be safe and effective when used in topical ophthalmic products.

In another embodiment the ophthalmic formulation further includes an osmolarity agent, such as sodium chloride and glycerine.

In another embodiment the ophthalmic formulation further includes a buffering agent selected from a citrate, acetate, phosphate, maleate, and other pharmaceutically acceptable buffer, singly or in combination at levels that are not irritating or discomforting to the eye. In another embodiment the ophthalmic formulation further includes a suspending agent selected from carboxymethylcellulose sodium (CMC), hydroxyethylcellulose, hypromellose, polyvinyl alcohol, povidone, carbomers, hyaluronic acid and its salts, chondroitin sulfate and its salts, natural gums, and other pharmaceutically acceptable polymers.

In another embodiment the ophthalmic formulation according to claim 1 further includes glycine as a stabilizer.

In one embodiment the pH of the ophthlamic formulation is between 3.0 and 7.0. In another embodiment the pH of the formulation is between 4.5 and 5.5. In a further embodiment the pH of the formulation is between about 5.0 and 5.2.

In one embodiment the A₁ agonist present in the ophthalmic formulation is between 0.05-5.0%, w/v. In one embodiment the surfactant present in the ophthalmic formulation is between 0.2-0.5%, w/v. In another embodiment the preservative present in the ophthalmic formulation is between 0.005-0.015%, w/v.

In one embodiment the ophthalmic formulation comprises:

| Ingredient | %, w/v |
|---|---|
| An A1 agonist (e.g, A-H), micronized | 0.05-5.0 |
| A suspending agent | 0.4-1.5 |
| A preservative | 0.005-0.015 |
| A surfactant | 0.2-0.5 |
| A buffering agent | 5 mM-20 mM |
| Glycine | 0-0.2 |
| NaCl | TBD (qs to 270-330 mOsm) |
| NaOH/HCl (pH adjustment) | pH 3.0-7.0 ± 0.1 |
| Purified Water | q.s. 100.00. |

In one embodiment the ophthalmic formulation comprises

| Ingredient | %, w/v |
|---|---|
| Compound A, micronized | 0.4 |
| Sodium CMC, low viscosity | 0.70 |
| Benzalkonium Chloride | 0.01 |
| Polysorbate 80 | 0.3 |
| Citric Acid Monohydrate | 0.15-0.3 (7 mM-14 mM) |
| Glycine | 0-0.10 |
| NaCl | TBD (qs to 290-300 mOsm) |
| NaOH/HCl(pH adjustment) | pH 5.1 ± 0.1 |
| Purified Water | q.s. 100.00. |

In another embodiment the ophthalmic formulation comprises:

| Ingredient | %, w/v |
|---|---|
| Compound A, micronized | 0.4 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 290-300 mOsm) |
| NaOH/HCl(pH adjustment) | pH 5.1 ± 0.1 |
| Purified Water | q.s. 100.00. |

In one embodiment the ophthalmic formulation comprises

| Ingredient | %, w/v |
|---|---|
| Compound A, micronized | 2.0 |
| Sodium CMC, low viscosity | 0.70 |
| Benzalkonium Chloride | 0.01 |
| Polysorbate 80 | 0.3 |
| Citric Acid Monohydrate | 0.15-0.3 (7 mM-14 mM) |
| Glycine | 0-0.10 |
| NaCl | TBD (qs to 290-300 mOsm) |
| NaOH/HCl(pH adjustment) | pH 5.1 ± 0.1 |
| Purified Water | q.s. 100.00. |

In one embodiment the ophthalmic formulation comprises:

| Ingredient | %, w/v |
|---|---|
| Compound A, micronized | 0.152 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

In one embodiment the ophthalmic formulation comprises:

| Ingredient | %, w/v |
|---|---|
| Compound A, micronized | 0.30 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

In one embodiment the ophthalmic formulation comprises:

| Ingredient | %, w/v |
|---|---|
| Compound A, micronized | 0.61 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

In one embodiment the ophthalmic formulation comprises:

| Ingredient | %, w/v |
|---|---|
| Compound A, micronized | 0.91 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

In one embodiment the ophthalmic formulation comprises:

| Ingredient | %, w/v |
|---|---|
| Compound A, micronized | 2.42 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

In a further aspect the present invention provides a method of reducing intraocular pressure comprising the step of: applying an effective amount of an ophthalmic formulation as described above to an affected eye of a subject. In one embodiment the ophthalmic formulation is administered to the affected eye of the subject in 30-50 μl drops. In another embodiment the ophthalmic formulation is administered in 1 to 2 drops once or twice daily. In another embodiment the subject has normal-tension glaucoma, OHT, or POAG.

In a further aspect there is provided a process for preparing an ophthalmic formulation as described above comprising the steps of

(a) milling the A₁ agonist form into particle sizes of less than about 50 microns;

(b) sterilizing the milled A₁ agonist; and

(c) aseptically suspending the particles of the A₁ agonist in an aqueous suspension comprising a surfactant and a preservative.

In one embodiment the milled A₁ agonist is sterilized by gamma irradiation up to a maximum of 40 Gray (Gy).

In another embodiment the process includes the further step of adjusting the pH of the aqueous suspension to between 3.0 and 7.0.

In a further embodiment the concentration of the A₁ agonist in the suspension is adjusted to between 0.05 and 5.0% (w/v), or in another embodiment the A₁ agonist in the suspension is adjusted to about 0.3 to 2.0% (w/v).

In one embodiment the concentration of the A1 agonist in the suspension is between 1-50 mg/ml, or in another embodiment the A1 agonist in the suspension is between 3 to 30 mg/ml.

The foregoing brief summary broadly describes the features and technical advantages of certain embodiments of the present invention. Further technical advantages will be described in the detailed description of the invention that follows. Novel features which are believed to be characteristic of the invention will be better understood from the detailed description of the invention when considered in connection with any accompanying figures and examples. However, the figures and examples provided herein are intended to help illustrate the invention or assist with developing an understanding of the invention, and are not intended to be definitions of the invention's scope.

### BRIEF DESCRIPTION OF DRAWINGS

FIG. 1 shows the effect on IOP in Dutch-Belted rabbits of three placebo ophthalmic formulations not including the A1 agonist compound A over time prior to and post administration.

FIG. 2 shows the effect on IOP in Dutch-Belted rabbits of three ophthalmic formulations including the A1 agonist compound A over time prior to and post administration.

### DETAILED DESCRIPTION

### Definitions

The term “surfactant” refers to a soluble compound that reduces the surface tension of liquids, or reduces interfacial tension between two liquids or a liquid and a solid, the surface tension being the force acting on the surface of a liquid, tending to minimize the area of the surface. Surfactants are used in pharmaceutical formulations in order to modify the absorption of the drug or its delivery to the target tissues. Well known surfactants include polysorbates (Polyoxyethylene derivatives; Tween) as well as Pluronic. In one embodiment the surfactant is selected from polysorbate 80, polysorbate 60, polysorbate 40, polysorbate 20, polyoxyl 40 stearate, poloxamers, tyloxapol, POE 35 and castor oil.

The term “preservative” refers to a compound in a pharmaceutical formulation that acts as an anti-microbial agent. In one embodiment the preservative is selected from a quaternary ammonium salts including benzalkonium chloride, cetrimide, chlorobutanol, sorbic acid, boric acid, and any other preservatives known to be safe and effective when used in topical ophthalmic products.

The term “topical application” as used herein means application by way of a liquid, gel or ointment to the external corneal surface of a subject.

The term “subject” means a human subject or an animal subject.

The term “effective amount” as used herein refers to an amount of an ophthalmic formulation that is effective for: (i) treating or preventing elevated IOP; or (ii) reducing IOP in a subject.

The particle size of the “fine particles” which may be used in the invention is preferably not more than 50 micron, which is about the maximum particle size tolerated ophthalmically in topical formulations. The particle size may be between 1-50 microns, e.g., less than 50 microns, less than 40 microns, less than 30 microns, less than 20 microns, or less than 10 microns. In one embodiment, a “fine particle” can also be referred to as a “micronized” particle.

As used herein, the term “drop” refers to a quantity of ophthalmically acceptable fluid that resembles a liquid drop. In one embodiment, a drop refers to a liquid volume equivalent to about 5 μl to about 200 μl, e.g., about 30 μl to about 80 μl, e.g., about 50 μl.

The following abbreviations are used herein and have the indicated definitions: IOP is intraocular pressure; OHT is ocular hypertension or POAG is primary open-angle glaucoma; HPβCD is hydroxypropyl β-cyclodextrin; sodium CMC is sodium carboxymethylcellulose.

### Fine Particle Compounds

The A₁ agonist in the present suspension of a fine particle is selective to the adenosine A₁ receptor and includes, but is not limited to ((2R,3S,4R,5R)-5-(6-(cyclopentylamino)-9H-purin-9-yl)-3,4-dihydroxytetrahydrofuran-2-yl)methyl nitrate;
((2R,3S,4R,5R)-5-(2-chloro-6-(cyclopentylamino)-9H-purin-9-yl)-3,4-dihydroxytetrahydrofuran-2-yl)methyl nitrate;
sodium ((2R,3S,4R,5R)-5-(6-(cyclopentylamino)-9H-purin-9-yl)-3,4-dihydroxytetrahydrofuran-2-yl)methyl sulfate;
((2R,3S,4R,5R)-3,4-dihydroxy-5-(6-(tetrahydrofuran-3-ylamino)-9H-purin-9-yl)tetrahydrofuran-2-yl)methyl nitrate;
((2R,3S,4R,5R)-5-(6-(cyclohexylamino)-9H-purin-9-yl)-3,4-dihydroxytetrahydrofuran-2-yl)methyl nitrate;
((2R,3S,4R,5R)-5-(6-(bicycle-[2.2.1]-heptan-2-ylamino)-9H-purin-9-yl)-3,4-dihydroxytetrahydrofuran-2-yl)methyl nitrate;
sodium ((2R,3S,4R,5R)-5-(2-chloro-6-(cyclohexylamino)-9H-purin-9-yl)-3,4-dihydroxytetrahydrofuran-2-yl)methyl sulfate; and
((2R,3S,4R,5R)-5-(2-chloro-6-(cyclohexylamino)-9H-purin-9-yl)-3,4-dihydroxytetrahydrofuran-2-yl)methyl nitrate.

Here and elsewhere, where discrepancies exist between a compound's name and a compound's structure, the chemical structure will control.

Compounds that act as selective adenosine A1 agonists are known and have shown a variety of utilities. U.S. Pat. Nos. 7,423,144 and 7,732,424 to Jagtap et al. describe the synthesis of certain A1 agonists, as does co-pending application U.S. Ser. No. 12/771,289. These references are incorporated herein by reference in their entireties.

The process for preparing the fine particles of the A1 agonist may be carried out with any of breakdown process such as a ball mill, a bead mill, a jet mill, and a hammer mill; spray dry; and built-up process such as crystallization.

The surfactant of the invention is used as a wetting or dispersing agent to disaggregate the particles of the micronized A₁ agonist into its micronized particles and to wet the surfaces of the particles to maintain compatibility with the aqueous solution. The surfactant is selected from the group of surface active agents that are primarily nonionic and include without limitation polysorbate 80, polysorbate 60, polysorbate 40, polysorbate 20, polyoxyl 40 stearate, poloxamers, tyloxapol, POE 35 and castor oil. It is to be appreciated that any similar pharmaceutically acceptable surface active agents may be usable at levels that do not cause irritation or discomfort when applied to the eye as topical drops. The preservative of the invention is used to preserve the ophthalmic formulation upon storage and is required for multi-dose ophthalmic formulations. Suitable preservatives include quaternary ammonium salts such as benzalkonium chloride, cetrimide, chlorobutanol, sorbic acid, boric acid, and any other preservatives known to be safe and effective when used in topical ophthalmic products. The antimicrobial efficacy might be enhanced, especially with the quaternary ammonium salts, by the addition of chelating agents such as edetate disodium.

Suspending agents are used to increase the viscosity and reduce the settling rate of the micronized particles in suspension and to allow for uniform dosing by an end user. Suspending agents help to ensure uniformity in the manufacturing and filling processes. Suspending agents are primarily polymers that are synthetic, semi-synthetic, or natural, and include without limitation: soluble cellulose derivatives such as carboxymethylcellulose sodium (CMC), hydroxyethylcellulose, hypromellose and others; polyvinyl alcohol, povidone, carbomers, hyaluronic acid and its salts, chondroitin sulfate and its salts, natural gums, and other pharmaceutically acceptable polymers. It is important to note that these suspending agents might also provide some surfactant properties as noted above.

Buffering agents are used to maintain the pH during shelf life in the range most optimum to maintain chemical stability of the micronized particles of the A1 agonist. Suitable buffering agents include citrates, acetates, phosphates, maleates, and other pharmaceutically acceptable buffers, singly or in combination at levels that are not irritating or discomforting to the eye.

In one embodiment, provided herein is an ophthalmic formulation for topical application comprising a micronized A₁ agonist (e.g., compound A), a suspending agent, a preservative, a surfactant, a buffering agent, glycine, and NaCl. The formulation can optionally include NaOH and HCl for pH adjustment, and/or purified water. The A₁ agonist content in the ophthalmic formulation of the invention is between 0.05 to 5% (w/v), or in one embodiment 0.3 to 2.0% (w/v). In another embodiment, provided herein is an ophthalmic formulation for topical application comprising micronized compound A, low viscosity sodium CMC, benzalkonium chloride, polysorbate 80, citric acid monohydrate, glycine, and NaCl. The formulation can optionally include NaOH and HCl for pH adjustment, and/or purified water.

### Method of Treatment

As described in the co-pending application U.S. Ser. No. 12/771,289, drug therapies that have proven to be effective for the reduction of intraocular pressure include both agents that decrease aqueous humor production and agents that increase the outflow facility. Such therapies are in general administered by one of two possible routes: topically (direct application to the eye) or orally. However, pharmaceutical ocular anti-hypertension approaches have exhibited various undesirable side effects. For example, miotics such as pilocarpine can cause blurring of vision, headaches, and other negative visual side effects. Systemically administered carbonic anhydrase inhibitors can also cause nausea, dyspepsia, fatigue, and metabolic acidosis. Certain prostaglandins cause hyperemia, ocular itching, and darkening of eyelashes and periorbital tissues. Further, certain beta-blockers have increasingly become associated with serious pulmonary side-effects attributable to their effects on beta-2 receptors in pulmonary tissue. Sympathomimetics cause tachycardia, arrhythmia and hypertension. Such negative side-effects may lead to decreased patient compliance or to termination of therapy such that normal vision continues to deteriorate. Additionally, there are individuals who simply do not respond well when treated with certain existing glaucoma therapies.

It has now been found that a selective adenosine A1 agonist in fine particle form reduces IOP in humans in clinical studies. In particular, formulations described herein are compounds of Formula I (e.g., Compounds A, B, C, D, E, F, G or H), in fine particle form, that can reduce intraocular pressure in a subject (e.g., a human) in need thereof. As shown in copending application U.S. Ser. No. 12/771,289 Compound A has been found to induce a statistically significant dose-related decrement in IOP upon a single application of Compound A to the eye of a human exhibiting elevated intraocular pressure as a result of OHT or POAG. Compound A formulated in 1 part to 20 parts Hydroxypropyl β-Cyclodextrin (HPβCD) (i.e. 1:20 wt/wt) reconstituted with 0.9% Saline for Injection, USP, was administered to one eye per subject (the study eye). Dosages ranged from 2.5 micrograms to 700 micrograms per respective treatment group. The 350 mcg and 700 mcg treatment groups showed the greatest reduction in IOP.

### EXAMPLES

The present invention is further illustrated by the following examples, but should not be construed to be limited thereto.

### Preparation Example

The invention provides an ophthalmic formulation comprising an aqueous suspension of fine particles of an A1 agonist. The A1 agonist, e.g., Compound A, in API form was fed into a loop mill at the rate of between 50-70 gms per hour and at a mill pressure of 90 psi. The milling process produced fine particles having a range of particle sizes of between 3-7 microns with an average particle size of about 5 microns. It is generally recognized that particle sizes less than 50 microns can be administered topically to the cornea in an ophthalmic formulation without undue irritation to the cornea or ocular tissue. Once Compound A was milled the resulting fine particles were sterilized by a gamma irradiation process. The particles were irradiated at up to 40 Gray (Gy) to sterilize the Compound A.

### Formulation Preparation

The suspension batches of Compound A were made at Newport Research in California at room temperature and atmospheric pressure and the batches ranged in volume from 10 mL to 120 mL and in concentration from 0.4% to 2.5% of Compound A. Most batches required the use of a stator-rotor mixer (a high-shear mixer) to provide enough shear to achieve adequate wetting and dispersion of the Compound A aggregates to the primary micronized particles. The specific mixer used was an OMNI MIXER HOMOGENIZER, Model 17105 with 10 mm generator probe for 10 mL batches and 20 mm generator probe for batches of 100-120 mL. Batches of 20 mL were prepared by ultrasonication for about 20-30 minutes and that was found to be sufficient for adequate dispersion as determined by microscopic examination.

The steps taken for manufacturing the sterile formulations were as follows:
1. Carboxymethylcellulose sodium (CMC) was dissolved in about 70%-90% of water in a batch. Purified Water (or Water for Injection) may be used. This step may be done using warm (50° C.-70° C.) or room temperature water.
2. Citric acid was added to the CMC and mixed to dissolve.
3. The pH was adjusted to 5.1±0.1 with sodium hydroxide solution of appropriate strength.
4. Polysorbate 80 was added and mixed. The mixing was gentle to avoid foaming.
5. Benzalkonium chloride was then added and mixed gently to avoid foaming.
6. Sodium chloride was then added and mixed so as to avoid foaming.
7. The pH was measured and re-adjusted again to 5.1±0.1 if necessary.
8. The resulting solution was filtered through a 0.2-micron sterilizing filter in a sterile manufacturing tank equipped with a powder transfer device and suitable mixer(s). Prefilters of larger pore sizes such as 5 or 20 micron might be used if necessary.
9. The sterile and micronized particles of Compound A were then added aseptically to the solution resulting from Step 8 and then mixed to achieve complete wetting/dispersion of the micronized particles of Compound A.
10. The balance of the water in the batch was then added and mixed to ensure homogeneity.
11. Lastly, the final pH was measured and adjusted if necessary to a pH of 5.1 with sodium hydroxide or hydrochloric acid.

### Formulation Example 1

The following Formulation was prepared according to the Formulation Preparation Example described above, however, glycine was added after the citric acid and the pH was adjusted with hydrochloric acid.

| Ingredient | %, w/v |
|---|---|
| Compound A, micronized | 0.4 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.1 |
| NaCl | 0.8 (qs to 290-300 mOsm) |
| NaOH/HCl | pH 5.1 ± 0.1 |
| Purified Water | q.s. 100.00. |

### Formulation Example 2

The following Formulation was prepared according to the Formulation Preparation Example described above.

| Ingredient | %, w/v |
|---|---|
| Compound A, micronized | 0.4 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 290-300 mOsm) |
| NaOH/HCl | pH 5.1 ± 0.1 |
| Purified Water | q.s. 100.00. |

### Formulation Example 3

The following Formulation was prepared according to the Formulation Preparation Example described above, however, glycine was added after the citric acid and the pH was adjusted with hydrochloric acid.

| Ingredient | %, w/v |
|---|---|
| Compound A, micronized | 0.4 |
| Sodium CMC, low viscosity | 0.70 |
| Benzalkonium Chloride | 0.01 |
| Polysorbate 80 | 0.3 |
| Citric Acid Monohydrate | 0.3 (14 mM) |
| Glycine | 0.1 |
| NaCl | 0.8 (qs to 290-300 mOsm) |
| NaOH/HCl | pH 5.1 ± 0.1 |
| Purified Water | q.s. 100.00. |

### Formulation Example 4

The following Formulation was prepared according to the Formulation Preparation Example described above, however, glycine was added after the citric acid and the pH was adjusted with hydrochloric acid.

| Ingredient | %, w/v |
|---|---|
| Compound A, micronized | 2.0 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.1 |
| NaCl | 0.8 (qs to 290-300 mOsm) |
| NaOH/HCl | pH 5.1 ± 0.1 |
| Purified Water | q.s. 100.00. |

### Formulation Example 5

A batch of Clinical Trial Material (CTM) was prepared under sterile conditions using the same procedure as described above for the Formulation Preparation Example and made up according to the following Formulation.

| Ingredient | %, w/v |
|---|---|
| Compound A, micronized | 0.152 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

### Formulation Example 6

A batch of Clinical Trial Material (CTM) was prepared under sterile conditions using the same procedure as described above for the Formulation Preparation Example and made up according to the following Formulation.

| Ingredient | %, w/v |
|---|---|
| Compound A, micronized | 0.30 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

### Formulation Example 7

A batch of Clinical Trial Material (CTM) was prepared under sterile conditions using the same procedure as described above for the Formulation Preparation Example and made up according to the following Formulation.

| Ingredient | %, w/v |
|---|---|
| Compound A, micronized | 0.61 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

### Formulation Example 8

A batch of Clinical Trial Material (CTM) was prepared under sterile conditions using the same procedure as described above for the Formulation Preparation Example and made up according to the following Formulation.

| Ingredient | %, w/v |
|---|---|
| Compound A, micronized | 0.91 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

### Formulation Example 9

A batch of Clinical Trial Material (CTM) was prepared under sterile conditions using the same procedure as described above for the Formulation Preparation Example and made up according to the following Formulation.

| Ingredient | %, w/v |
|---|---|
| Compound A, micronized | 2.42 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

### Use of Formulation Examples

### Animal Studies

The Formulation Examples 1 to 3 above were used in an animal study involving ocular normotensive Dutch-Belted rabbits. Additionally 3 equivalent placebo formulations to that of Formulation Examples 1, 2 and 3 described above were prepared as per the Formulation Examples 1, 2 and 3 above, but lacking Compound A. Ocular normotensive Dutch-belted rabbits were prepared for study and for each treatment group nine (n=9) animals were chosen. In each treatment group 6 animals received a single 50 μl dose topically to the cornea of one treatment eye of the Formulation Example, while the remaining 3 animals received the respective placebo topically to the cornea of one treatment eye. The intraocular pressure of the treatment eye of each of the rabbits in each treatment group was measured at time intervals, 1, 2, 4, 6 and 8 hours post administration of either the Formulation Example or the respective placebo.

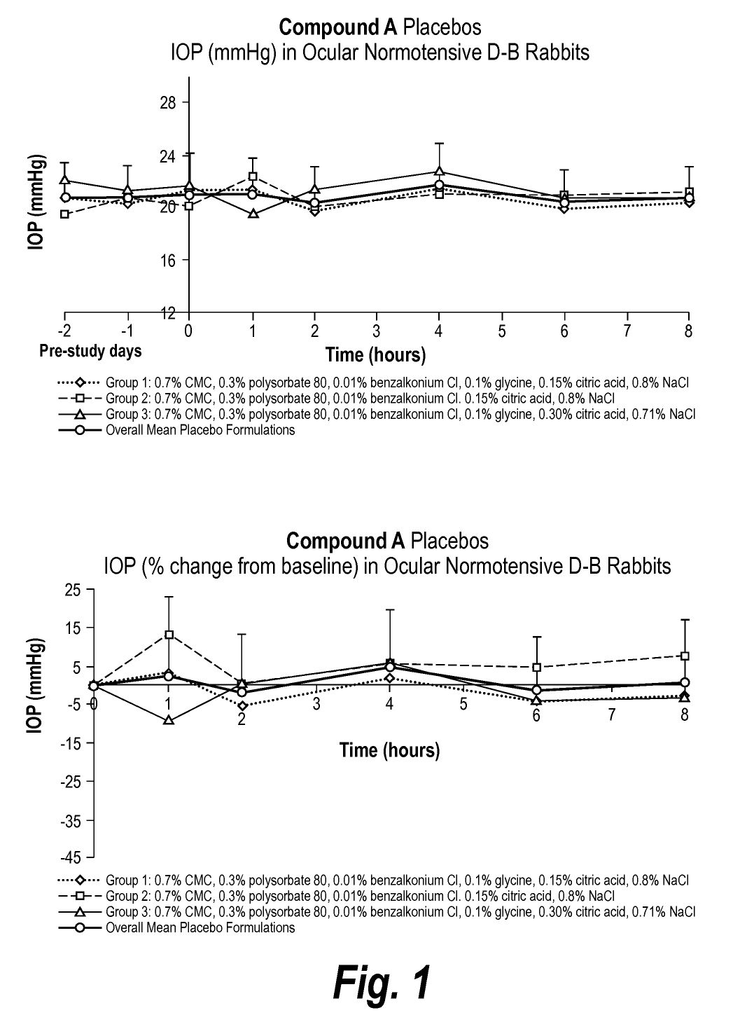

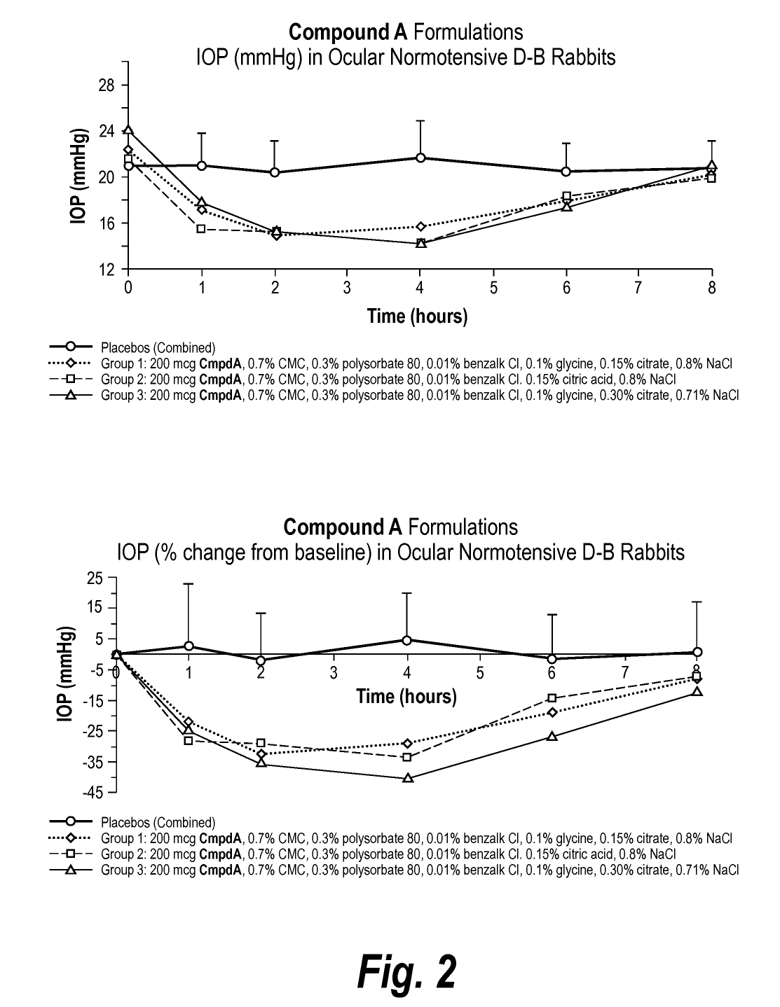

The results of the study are shown in FIGS. 1 and 2. It can be seen in the plots shown in FIG. 1 that the IOP values seen before and after administration of the respective placebos are comparable to baseline levels.

It can further be seen in the plots shown in FIG. 2 that the IOP values at and post administration of the respective Formulation Examples were reduced from baseline levels over the 8 hour period post administration. Furthermore it can be seen that all three Formulation Examples produced comparable reductions in IOP. It is anticipated that similar results would be seen in the reduction of IOP in humans given the earlier clinical trial completed by the Applicant as described in co-pending application U.S. Ser. No. 12/771,289.

### Stability Studies

The formulation prepared in Formulation Example 4 was studied for stability over a 3 month period at 2-8 degrees C. Samples were taken at 2 weeks, 1 month, 2 months, 3 months, 6 months and 12 months and analyzed by liquid chromatography. The stability findings are tabulated in Table 1 below.

TABLE 1

| Time at 2-8° C. | | Compound A % | Impurities % | pH |
|---|---|---|---|---|
| 0 | | 100.00 | 0.28 | 4.9 |
| 2 | weeks | 100.99 | 0.25 | 4.87 |
| 1 | month | 100.11 | 0.29 | 4.88 |
| 2 | months | 98.21 | 0.22 | 4.91 |
| 3 | months | 98.79 | 0.23 | 4.92 |
| 6 | months | 96.590 | 0.21 | 4.89 |
| 12 | months | 97.423 | 0.17 | 4.83 |

As can be seen from the results in Table 1 the suspension formulation of Formulation Example 4 was substantially stable over a 12 month period upon storage at 2-8° C.

The formulation prepared in Formulation Example 2 was studied for stability over a 6 month period at 2-8 degrees C. Samples were taken at 1 month, 2 months, 3 months, 4 months and 6 months and analyzed by liquid chromatography. The stability findings are tabulated in Table 1 below.

TABLE 2

| Time at 2-8° C. | | Compound A % | Impurities % | pH |
|---|---|---|---|---|
| 0 | | 100 | 0.28 | 5.17 |
| 1 | month | 97.8 | 0.23 | 5.04 |
| 2 | months | 102.4 | 0.24 | 5.16 |
| 3 | months | 101.8 | 0.3 | 5.21 |
| 4 | months | 105.2 | 0.3 | 5.20 |
| 6 | months | 106.5 | 0.34 | 5.09 |
| 12 | months | 102.9 | 0.41 | 5.18 |

As can be seen from the results in Table 2 the suspension formulation of Formulation Example 2 was substantially stable over a 12 month period upon storage at 2-8° C.

At the time of filing this application, the stability of Formulation Examples 5-9 had been studied for a period of 3 months under refrigerated conditions (5 degrees Celsius) and the stability of these Formulation Examples appeared satisfactory, with the pH readings remaining stable and the percentage of total impurities remaining substantially unchanged.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

What is claimed is:

1. An ophthalmic formulation for topical application comprising:
(a) an aqueous suspension of a fine particle of an A₁ agonist,
(b) a surfactant, and
(c) a preservative.

2. The ophthalmic formulation according to claim 1, wherein the suspension of an A1 agonist comprises fine particles of less than 50 microns.

3. The ophthalmic formulation according to claim 2, wherein the fine particles are less than 10 microns.

4. The ophthalmic formulation according to claim 2, wherein the fine particles are between 3-7 microns.

5. The ophthalmic formulation according to claim 1, wherein the A1 agonist is selected from: 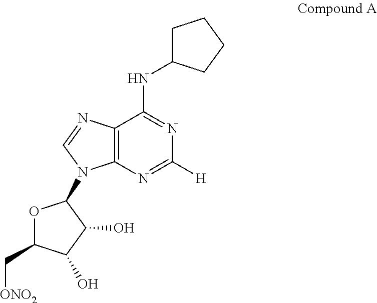
((2R,3S,4R,5R)-5-(6-(cyclopentylamino)-9H-purin-9-yl)-3,4-dihydroxytetrahydrofuran-2-yl)methyl nitrate, 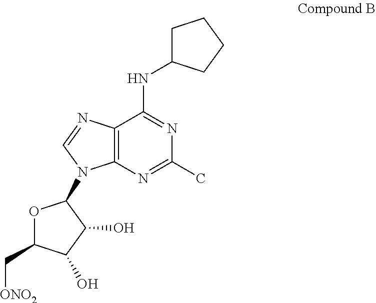
((2R,3S,4R,5R)-5-(2-chloro-6-(cyclopentylamino)-9H-purin-9-yl)-3,4-dihydroxytetrahydrofuran-2-yl)methyl nitrate, 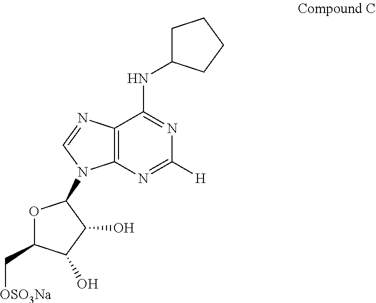
sodium ((2R,3S,4R,5R)-5-(6-(cyclopentylamino)-9H-purin-9-yl)-3,4-dihydroxytetrahydrofuran-2-yl)methyl sulfate, 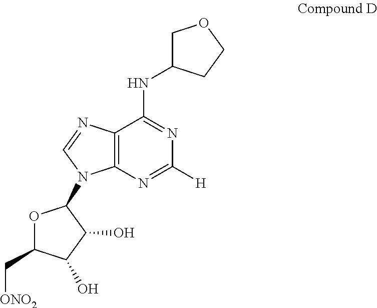
((2R,3S,4R,5R)-3,4-dihydroxy-5-(6-(tetrahydrofuran-3-ylamino)-9H-purin-9-yl)tetrahydrofuran-2-yl)methyl nitrate, 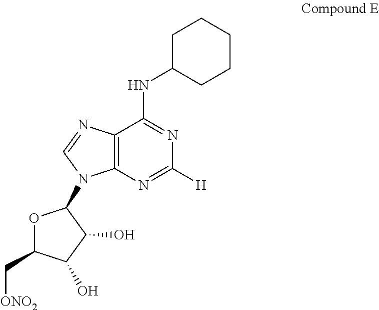
((2R,3S,4R,5R)-5-(6-(cyclohexylamino)-9H-purin-9-yl)-3,4-dihydroxytetrahydrofuran-2-yl)methyl nitrate, 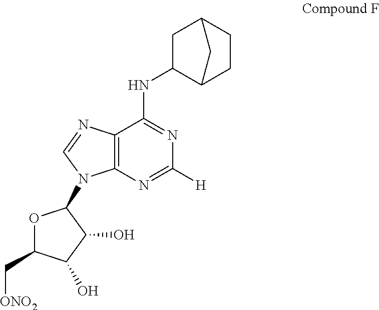
((2R,3S,4R,5R)-5-(6-(bicycle-[2.2.1]-heptan-2-ylamino)-9H-purin-9-yl)-3,4-dihydroxytetrahydrofuran-2-yl)methyl nitrate, 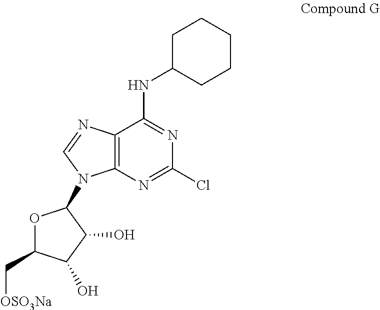
sodium ((2R,3S,4R,5R)-5-(2-chloro-6-(cyclohexylamino)-9H-purin-9-yl)-3,4-dihydroxytetrahydrofuran-2-yl)methyl sulfate, and 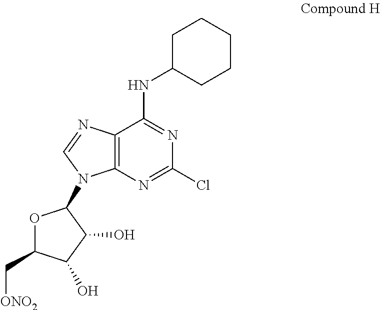
((2R,3S,4R,5R)-5-(2-chloro-6-(cyclohexylamino)-9H-purin-9-yl)-3,4-dihydroxytetrahydrofuran-2-yl)methyl nitrate.

6. The ophthalmic formulation according to claim 1, wherein the A1 agonist is compound A 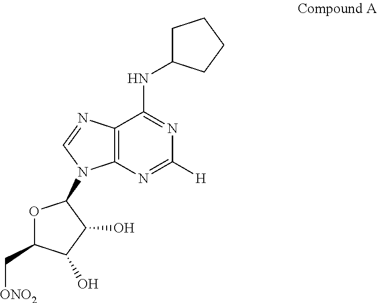
((2R,3S,4R,5R)-5-(6-(cyclopentylamino)-9H-purin-9-yl)-3,4-dihydroxytetrahydrofuran-2-yl)methyl nitrate.

7. The ophthalmic formulation according to claim 1, wherein the surfactant is selected from polysorbate 80, polysorbate 60, polysorbate 40, polysorbate 20, polyoxyl 40 stearate, poloxamers, tyloxapol, POE 35 and castor oil.

8. The ophthalmic formulation according to claim 1, wherein the preservative is selected from a quaternary ammonium salts including benzalkonium chloride, cetrimide, chlorobutanol, sorbic acid, boric acid, and any other preservatives known to be safe and effective when used in topical ophthalmic products.

9. The ophthalmic formulation according to claim 1, further including an osmolarity agent.

10. The ophthalmic formulation according to claim 9, wherein the osmolarity agent is selected from sodium chloride and glycerine.

11. The ophthalmic formulation according to claim 1, further including a buffering agent.

12. The ophthalmic formulation according to claim 11, wherein the buffering agent is selected from a citrate, acetate, phosphate, maleate, and other pharmaceutically acceptable buffer, singly or in combination at levels that are not irritating or discomforting to the eye.

13. The ophthalmic formulation according to claim 1, further including a suspending agent.

14. The ophthalmic formulation according to claim 1 wherein the suspending agent selected from carboxymethylcellulose sodium (CMC), hydroxyethylcellulose, hypromellose, polyvinyl alcohol, povidone, carbomers, hyaluronic acid and its salts, chondroitin sulfate and its salts, natural gums, and other pharmaceutically acceptable polymers.

15. The ophthalmic formulation according to claim 1 further including glycine as a stabilizer.

16. The ophthalmic formulation according to claim 1 wherein the pH of the formulation is between 3.0 and 7.0.

17. The ophthalmic formulation according to claim 16 wherein the pH of the formulation is between about 4.5 and 5.5.

18. The ophthalmic formulation according to claim 17 wherein the pH of the formulation is between about 5.0 and 5.2.

19. The ophthalmic formulation according to claim 1 wherein the A₁ agonist present in the formulation is between 0.05-5.0%, w/v.

20. The ophthalmic formulation according to claim 1 wherein the surfactant present in the formulation is between 0.2-0.5%, w/v.

21. The ophthalmic formulation according to claim 1 wherein the preservative present in the formulation is between 0.005-0.015%, w/v.

22. The ophthalmic formulation according to claim 1 comprising:

| Ingredient | %, w/v |
|---|---|
| A1 agonist, micronized | 0.05-5.0 |
| A suspending agent | 0.4-1.5 |
| A preservative | 0.005-0.015 |
| A surfactant | 0.2-0.5 |
| A buffering agent | 5 mM-20 mM |
| Glycine | 0-0.2 |
| NaCl | TBD (qs to 270-330 mOsm) |
| NaOH/HCl (pH adjustment) | pH 3.0-7.0 ± 0.1 |
| Purified Water | q.s. 100.00. |

23. The ophthalmic formulation according to claim 1 comprising:

| Ingredient | %, w/v |
|---|---|
| Compound A, micronized | 0.4 (4 mg/ml) |
| Sodium CMC, low viscosity | 0.70 |
| Benzalkonium Chloride | 0.01 |
| Polysorbate 80 | 0.3 |
| Citric Acid Monohydrate | 0.15-0.3 (7 mM-14 mM) |
| Glycine | 0-0.10 |
| NaCl | TBD (qs to 290-300 mOsm) |
| NaOH/HCl (pH adjustment) | pH 5.1 ± 0.1 |
| Purified Water | q.s. 00.00. |

24. The ophthalmic formulation according to claim 1 comprising:

| Ingredient | %, w/v |
|---|---|
| Compound A, micronized | 0.4 (4 mg/ml) |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 290-300 mOsm) |
| NaOH/HCl (pH adjustment) | pH 5.1 ± 0.1 |
| Purified Water | q.s. 100.00 |

25. The ophthalmic formulation according to claim 1 comprising:

| Ingredient | %, w/v |
|---|---|
| Compound A, micronized | 2.0 (20 mg/ml) |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.1 |
| NaCl | 0.8 (qs to 290-300 mOsm) |
| NaOH/HCl (pH adjustment) | pH 5.1 ± 0.1 |
| Purified Water | q.s. 100.00 |

26. The ophthalmic formulation according to claim 1 comprising:

| Ingredient | %, w/v |
|---|---|
| Compound A, micronized | 0.152 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

27. The ophthalmic formulation according to claim 1 comprising:

| Ingredient | %, w/v |
|---|---|
| Compound A, micronized | 0.30 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

28. The ophthalmic formulation according to claim 1 comprising:

| Ingredient | %, w/v |
|---|---|
| Compound A, micronized | 0.61 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

29. The ophthalmic formulation according to claim 1 comprising:

| Ingredient | %, w/v |
|---|---|
| Compound A, micronized | 0.91 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

30. The ophthalmic formulation according to claim 1 comprising:

| Ingredient | %, w/v |
|---|---|
| Compound A, micronized | 2.42 |
| Sodium CMC, low viscosity | 0.70 |
| Benzalkonium Chloride | 0.01 |
| Polysorbate 80 | 0.3 |
| Citric Acid Monohydrate | 0.15 (7 mM) |
| Glycine | 0.0 |
| NaCl | 0.8 (qs to 270-300 mOsm) |
| NaOH/HCl | pH 5.1 ± 0.1 |
| Purified Water | q.s. 100.00. |

31. A method of reducing intraocular pressure comprising the step of: applying an effective amount of an ophthalmic formulation according to claim 1 to an affected eye of a subject.

32-34. (canceled)

35. A process for preparing an ophthalmic formulation according to claim 1 comprising the steps of
(a) milling the A₁ agonist form into particle sizes of less than about 50 microns;
(b) sterilizing the milled A₁ agonist; and
(c) aseptically suspending the particles of the A₁ agonist in an aqueous suspension comprising a surfactant and a preservative.

36-43. (canceled)
